(19) United States
(12) Patent Application Publication
(10) Pub. No.: US 2023/0079554 A1
(43) Pub. Date: Mar. 16, 2023

(54) SYSTEMS, DEVICES AND RELATED GARMENTS FOR ACTIVE TEMPERATURE REGULATION

(71) Applicant: Olive Branch Research Group LLC, Raleigh, NC (US)

(72) Inventor: Alan J. Clark, Marietta, GA (US)

(21) Appl. No.: 17/944,323

(22) Filed: Sep. 14, 2022

Related U.S. Application Data
(60) Provisional application No. 63/243,799, filed on Sep. 14, 2021.

(51) Int. Cl. A41D 13/005

(52) U.S. Cl. CPC A41D 13/005

(57) ABSTRACT

Thermal regulating units are provided. Each thermal regulating unit includes a first thermally conductive layer in contact with the skin of the body. The first thermally conductive layer is embedded with electrical wiring and a first passive thermometer sensor. A plurality of spaced apart thermoelectric coolers are provided on the first thermal conductivity layer. A second thermally conductive layer opposite the first layer is exposed to ambient air or fluid. The second thermally conductive layer includes embedded electrical wiring and a second passive thermometer sensor. A thermal insulating layer is provided between the first and second thermally conductive layers. The thermal insulating layer creates a thermal barrier therebetween and adheres the first and second thermally conductive layer together. The thermal regulating unit operates responsive to a controller to regulate the temperature of a user based on the feedback of the one or more thermal regulating units.

### CLAIM OF PRIORITY

The present application claims priority to U.S. Provisional Application No. 63/243,799 filed on Sep. 14, 2021, entitled Systems, Devices and Related Garments for Active Temperature Regulation, the contents of which is incorporated herein.

### FIELD

The present inventive concept generally relates to thermal regulating units and, in particular, thermal regulating units that are wearable and provide active heating and cooling to regulate a user's body temperature.

### BACKGROUND

In cold or hot climates, it may be desirable to provide heating or cooling for personal comfort to an individual. For example, personal heating or cooling may be desired during activities such as skiing, camping, hiking, fishing, hunting, working, athletic activities, military and the like. Furthermore, in some circumstances, a person may suffer from a condition that makes it difficult for him or her to heat and/or cool their body; for example, a person with Anhidrosis does not have the ability to sweat normally, a person suffering from heat stroke or hypothermia or the like. Similarly, Conventional garments for heating and cooling are available from various vendors. However, there is no active heating and/or cooling component in these garments. Improved devices and systems for heating and cooling a body are desired.

### SUMMARY

Some embodiments of the present inventive concept provide a thermal regulating unit that regulates temperature of a body when positioned next to thermal points on the body. The thermal regulating unit includes a first thermally conductive layer adjacent skin of the body and including embedded electrical wiring; a first passive thermometer sensor integrated with the first thermally conductive layer; a plurality of spaced-apart thermoelectric coolers on the first thermal conductivity layer; a second thermally conductive layer opposite the first thermally conductive layer, the second thermally conductive layer being exposed to ambient air or fluid and including embedded electrical wiring; a second passive thermometer sensor integrated with the second thermally conductive layer; and a thermal insulating layer between the first and second thermally conductive layers to create a thermal barrier therebetween and adhere the first and second thermally conductive layers together. The thermal regulating unit operates responsive to a controller to regulate the temperature of the body.

Related systems and garments are also provided herein.

### BRIEF DESCRIPTION OF THE DRAWINGS

FIG. 1 is a diagram illustrating a sideview cross-section of a thermal regulating unit according to some embodiments of the present inventive concept.

FIG. 2 is a diagram illustrating a front view of example garments including a plurality of thermal regulating units positioned on the garments according to some embodiments of the present inventive concept.

FIG. 3 is a diagram illustrating a front view of example garments including a plurality of thermal regulating units positioned on the garments coupled to a plurality of controllers according to some embodiments of the present inventive concept.

FIG. 4A is a block diagram illustrating passive temperature sensors and Peltier coolers in accordance with some embodiments of the present inventive concept.

FIG. 4B is a block diagram illustrating a control unit and battery in accordance with some embodiments of the present inventive concept.

FIG. 5A is a cross section of a thermal regulating unit in accordance with some embodiments of the present inventive concept.

FIG. 5B is a top view of a thermal regulating unit in accordance with some embodiments of the present inventive concept.

FIG. 6 is a basic block diagram illustrating a data processing system for use with the thermal regulating units according to some embodiments of the present inventive concept.

### DETAILED DESCRIPTION

The inventive concept now will be described more fully hereinafter with reference to the accompanying drawings, in which illustrative embodiments of the inventive concept are shown. This inventive concept may, however, be embodied in many different forms and should not be construed as limited to the embodiments set forth herein; rather, these embodiments are provided so that this disclosure will be thorough and complete and will fully convey the scope of the inventive concept to those skilled in the art. Like numbers refer to like elements throughout. As used herein, the term “and/or” includes any and all combinations of one or more of the associated listed items.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As discussed above, conventional garments and devices sold to control temperature do not have a control component, i.e. they lack an active heating and/or cooling component. Accordingly, some embodiments of the present inventive concept provide a thermal regulating unit that regulates the temperature of a body when worn close to the skin. As will be discussed in detail herein, the thermal regulating unit communicates with a controller to adjust the temperature of the body up and down. For example, in some embodiments, the temperature may be adjusted up or down 20 degrees or more.

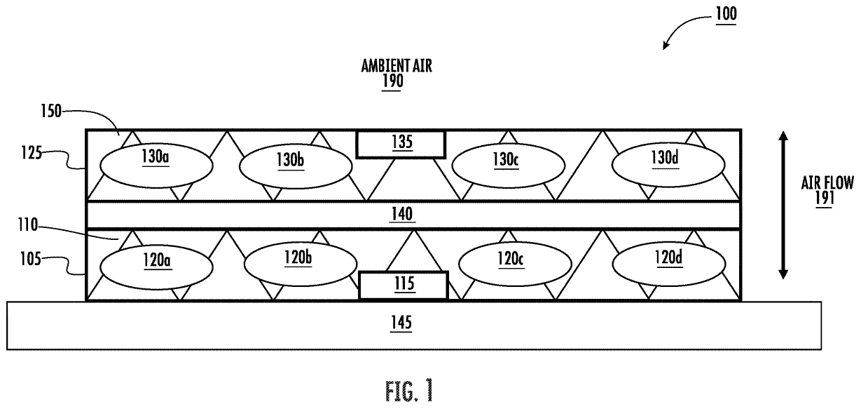

Referring first to FIG. 1, a cross-section of a thermal regulating unit 100 in accordance with some embodiments of the present inventive concept will be discussed. It will be understood that the cross-section of FIG. 1 is provided for example only and, therefore, embodiments of the present inventive concept are not limited to the embodiments illustrated therein. For example, FIG. 1 illustrates particular layers positioned in a pattern; however, other intervening layers may be present without departing from the scope of the present inventive concept. As illustrated in FIG. 1, the thermal regulating unit 100 includes first 105 and second 125 thermally conductive layers separated by a thermal insulating layer 140.

As shown, the first thermally conductive layer 105 on a first surface of the thermal insulating layer 140 is a non-electrically conductive layer in close proximity to, or possibly in contact with, the skin of the body 145 of the subject. The material selected for the first (and second) thermally conductive layer 105 can be any suitable thermally conductive material. However, in some embodiments, this material is relatively thin, waterproof, highly thermally conductive, durable, and non-electrically conductive. In some embodiments, the thermal conductively may be provided by a powder/dust, such as Carbene powder or diamond dust. Using a powder/dust allows the substance to be embedded into the material and may allow improved thermal conductivity. Embodiments of the present inventive concept are not limited to these examples.

As used herein, “waterproof” refers to the thermal regulating unit being capable of withstanding sweat produced by the body as well as being submerged underwater. Units being used for significant depths may be further reinforced to handle sustained submersion as well as the added pressure of depth. It will be understood that although the subject may be human, the subject may also be non-human and be used for other living subjects in need of thermal regulation. In some embodiments, the thermal regulating unit can withstand being submerged 40,000 feet or more and still be functional.

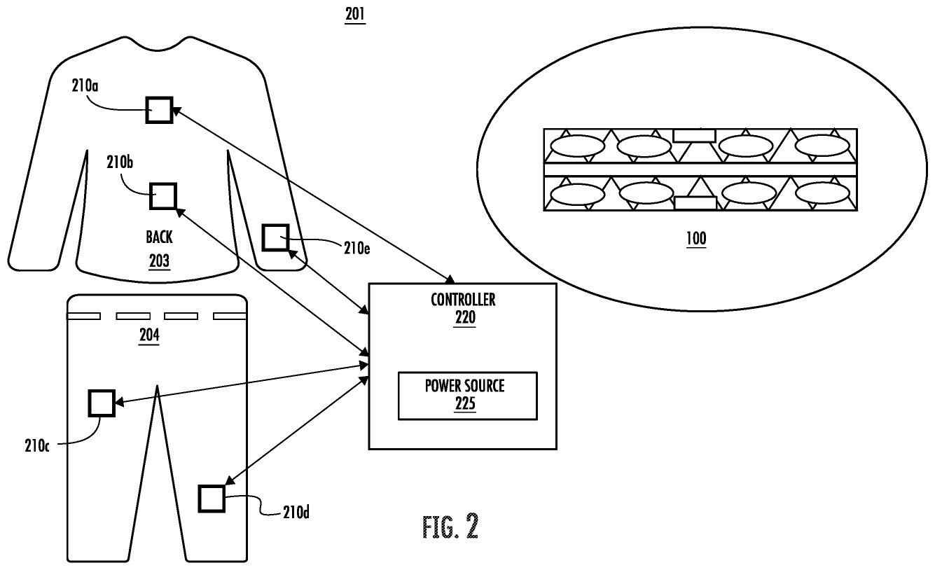

As further illustrated in FIG. 1, the first thermally conductive layer 105 includes electrical wiring 110 and one or more spaced-apart thermoelectric coolers 120a-120d, for example, Peltier coolers coupled to the electrical wiring 110. It will be understood that the electrically wiring may be embedded within the first thermally conductive layer or in a separate layer without departing from the scope of the present inventive concept. The first thermally conductive layer 105 also includes a first passive thermometer sensor 115. The first passive thermometer sensor 115 is provided to convert heat energy into electricity/power that is used to heat or cool the body as discussed herein. The first passive thermometer sensor 115 may also communicate with a controller (FIG. 2) to enable efficient thermal regulation of the body.

Embodiments of the present inventive concept use thermoelectric coolers (Peltier coolers) in combination with thermal conducting materials to draw heat from a larger area and reduce, or possibly minimize, the power consumption required by the cooling components. The coolers are spaced apart based on the thermal conductivity of the thermal conducting material.

The second thermally conductive layer 125 is provided on a surface of the thermal insulating layer 140 opposite the first thermally conductive layer 105. As shown, unlike the first thermally conductive layer 105, the second thermally conductive layer 125 is exposed to ambient air 190 or water/fluid in some embodiments. Similar to the first thermally conductive layer 105, the second thermally conductive layer 125 is a non-electrically conductive layer including embedded electrical wiring 150 and one or more spaced apart thermoelectric coolers 130a-130d coupled to the electrical wiring 150. A second passive thermometer sensor 135 is provided and performs the same or similar functions discussed above with respect to the first passive thermometer sensor 115

As illustrated, the thermal insulating layer 140 is provided between the first thermally conductive layer 105 and the second thermally conductive layer 125. The presence of the thermal insulating layer 140 between the first and second conductive layers provides a thermal barrier between the first and second thermally conductive layers and, therefore, protects against bleed over of heat to the cooled or heated side of the thermal regulation unit 100. The thermal insulating layer 140 may also be used to secure the thermoelectric coolers 120a-120d and 130a-130d to the first and second thermally conductive layers 105 and 125, respectively.

The first 105 and second 125 thermally conductive layers are sealed together to protect the layers therebetween and provide a thermal regulation unit 100 in accordance with some embodiments of the present inventive concept. The thermal regulation unit 100 is a flexible, thin, and lightweight wearable unit that regulates the temperature of a body. For example, the thermal regulation units should be flexible enough to be worn on clothing and wrap around large muscle groups, such as the legs and back. Thermal regulating units 100 in accordance with embodiments discussed herein may be used, for example, for military, sports and/or daily wear. In particular, in military garments, the thermal regulating unit 100 may be incorporated into, for example, dragon scale ballistic armor. This may allow for slightly better ballistics protection. Furthermore, the size, flexibility, and minimal weight of the thermal regulating unit 100 may allow for more mobility and less fatigue for the subject wearing the unit (s).

As illustrated in FIG. 1, the flow of air 191 around the thermal regulation unit 100 is used to regulate the temperature of the body. In particular, more airflow translates to more heat transfer. In some embodiments, the thermal regulation unit 100 is configured to have grooves thereon to provide more surface area on which the air can flow.

Referring now to FIG. 2, diagrams illustrating a system where thermal regulating units 100 are positioned in garments to be worn such that the subject wearing the garments may be cooled and/or heated in accordance with embodiments discussed herein. As illustrated in FIG. 2, the garments include a back of a long sleeve shirt 203 and pants 204. As discussed above, one side of the temperature regulation unit 100 must be worn in close proximity to skin of the body if not in full contact therewith, and the other side of the temperature regulation unit 100 must be exposed to ambient air (water/fluid). Thus a form-fitting shirt 203 and pants 204 would facilitate contact better than a looser shirt and pants. Similarly, any material shirt 203 and pants 204 may be used without departing from the scope of the present inventive concept. Various wick-away materials as well as both cold gear and hot gear exist and can be used in combination with embodiments discussed herein.

In particular, the use of form-fitting clothing may ensure that the thermal regulating unit has uninterrupted contact with the skin in the appropriate thermal exchange areas. Form-fitting garments may also make it easier to attach the thermal regulation unit to the body.

Referring again to FIG. 2, a plurality of thermal regulating units 100 are positioned on each garment 203 and 204. For example, as shown, one or more of the thermal regulating units 100 may be positioned in the garment. For best performance, the thermal regulating units 100 may be positioned in the garment such that they are near major thermal points of the body, for example, where the veins are close to the skin. Some of these locations may include, for example, the middle of the upper back between the shoulder blades (210a), which is a high thermal exchange area. Furthermore, the thermal regulation units 100 may be worn so that they wrap around large muscle groups, such as the legs (210c) and back. However, it will be understood that the thermal regulating units may be worn anywhere on the body, for example, the arm (210e) and the lower leg (210d) without departing from the scope of the present inventive concept. Thus, the thermal regulating units 100 do not need to heat/cool the entire skin of the body; it just needs to regulate the temperature in strategic places on the body. It will be understood that these strategic places may change based on the subject.

In some embodiments, the thermal regulating unit 100 may be positioned in the form-fitting garment 203/204 using, for example, integrated pockets 210a-210e as show in FIG. 2 and/or fasteners, including Velcro and/or straps. In some embodiments, the integrated pockets 210a-210e may be mesh or vented to allow air flow. The integrated pockets 210a-210e may be used to allow the thermal regulating units to be removed and the garment to be washed. The garment itself may be machine washable, and the unit 100 may be hand washable in some embodiments.

Again, the thermal regulating units 100 are not positioned in pockets in all embodiments. In some embodiments, the thermal regulating units may be fastened to the garment using a fastener. As used herein, a “fastener” or “fastening means” refers to any means of attached the thermal regulating unit to the garment. Thus, although Velcro, straps, etc. are discussed herein, embodiments of the present inventive concept are not limited thereto. Thus, all labeled locations 210a-210e may be locations on the garment where thermal regulating units 100 are fastened and not disposed in pockets in some embodiments.

In some embodiments, the thermal regulating units 100 operate in response to a controller 220 to regulate the temperature of the body. The controller is associated with a power source 225 for supplying power to the thermal regulating units 100. In some embodiments, the controller 220 and power source 225 may be separate from the thermal regulating unit 100. For example, the controller 220 and power source 225 may be the size of a cell phone or smaller and may communicate with each of the plurality of thermal regulating units 100 to regulate the temperature of the body.

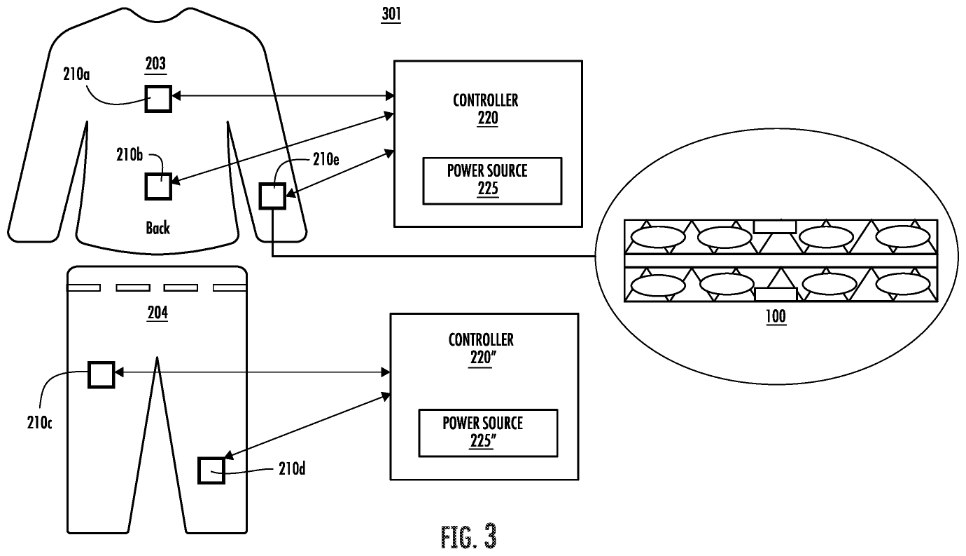

For example, the thermal regulating units 100 may provide feedback to the controller 220, based on signals provided from the first passive thermometer sensor 105 and/or second passive thermometer sensor 125 discussed above with respect to FIG. 1. The controller 220 manages the electricity/power supplied to the thermal regulating unit 100 based on the feedback. It will be understood that there may be more than one controller 220/power source 225 without departing from the scope of the present inventive concept as illustrated in, for example, FIG. 3.

In particular, FIG. 3 illustrates more than one controller 220 and 220″ to control a plurality of thermal control units 100 in pockets 210a-210e. For example, the thermal regulating units 100 in pockets 210a, 210b and 210e are coupled to a first controller 220, and may provide feedback to controller 220, based on signals provided from the first passive thermometer sensor 105 and/or second passive thermometer sensor 125 discussed above with respect to FIG. 1. Similarly, the thermal regulating units 100 in pockets 210c and 210d are coupled to a second controller 220″, and may provide feedback to the controller 220″, based on signals provided from the first passive thermometer sensor 105 and/or second passive thermometer sensor 125. The controllers 220 and 220″ manage the electricity/power supplied to each the thermal regulating units 100 coupled to each controller based on the feedback from each group of thermal regulating units 100. Although only two controllers are provided in FIG. 3, embodiments of the present inventive concept are not limited to this configuration. For example, three or more controllers may be provided without departing from the scope of the present inventive concept.

In some embodiments the controller 220, 220″ may be provided by a compact computing device. In some embodiments, this compact computing device may be provided by, for example, a computer similar to a gum stick computer, however, embodiments are not limited thereto. The controller 220, 220″ may be used to regulate power to the individual coolers; control the amount of thermal regulation required by user settings; monitor the efficiency of the thermal regulation component; and regulate and monitor safety. As discussed above, the controller 220, 220″ may be portable, compact and sealed. The controller 220, 220″ is designed to consume very little power.

Referring again to FIG. 2, in some embodiments, the power source 225 may be provided by a high-efficiency battery unit that stores electrical energy to be used by the thermal regulating unit 100. An external battery pack may also be provided. Given conventional battery technology, for example, lithium batteries, the cooling can last several hours on a single charge and may be greater for smaller cooling units. Wireless charging may also be provided for embedded battery packs. For military use, the battery pack may be placed on the side of the body, near the waist.

In some embodiments, the controller 220 regulates the temperature of the body using the thermal regulating unit 100 by increasing or decreasing the temperature of the thermal regulating unit in the range of 20 degrees Fahrenheit.

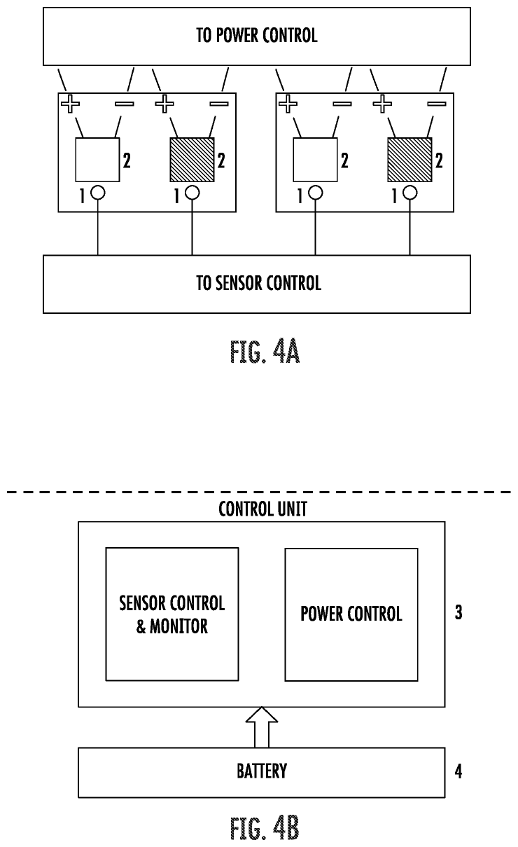

Further details of the controller or control unit including power and sensors will now be discussed with respect to FIGS. 4A and 4B. FIG. 4A illustrates a plurality of passive temperature sensors 1 each associated with a pair of coolers/heaters 2. These coolers/heaters 2 may be provided by Peltier coolers in some embodiments. These coolers may have both a “cool side” and a “hot side” to enable thermal regulating unit in accordance with embodiments discussed herein to regulate the temperature up and/or down. As illustrated, the temperature associated with each section (1, 2) may be increased (+) or decreased (−) based on the comfort of the user. As further illustrated in FIG. 4B, the sensors 1 send a signal to the power control 3 and a change is made responsive to the signal. The unit may be powered by a battery 4 as shown. The details of the battery 4 are discussed above with respect to FIG. 2.

As discussed above, some embodiments include a pair of Peltier devices for cooling and heating, one heats and one cools as discussed above. However, embodiments of the present inventive concept are limited to this configuration. One device may be provided that both cools and heats without departing from embodiments discussed herein. Furthermore, positioning the Peltier units would vary by embodiments and should be placed close enough to be effective but far enough apart to increase efficiency.

In some embodiments, the Peltier coolers/heaters will form “bumps” on a surface of the pad. This surface should be placed away from the skin in some embodiments.

It will be understood that the thermal regulating unit in accordance with embodiments discussed herein is waterproof. As discussed above, waterproof indicates that the unit will still operate if drenched in fluid, for example, sweat, water, etc. Thus, the unit will operate when the user is swimming, even if the water is deep.

In some embodiments, the thermal regulating unit should be thermally insulated on a side of the unit closest to the body of the user, but thermally conductive on the side spaced away from the body of the user to disperse heat. The power control may be adjusted based on information from the passive sensors. For example, if one passive sensor shows 0.25 Watts (heat) and another passive sensor shows 0.3 Watts (heat), cooling power to the first would be reduced and cooling power to the second would be increased to normalize the two.

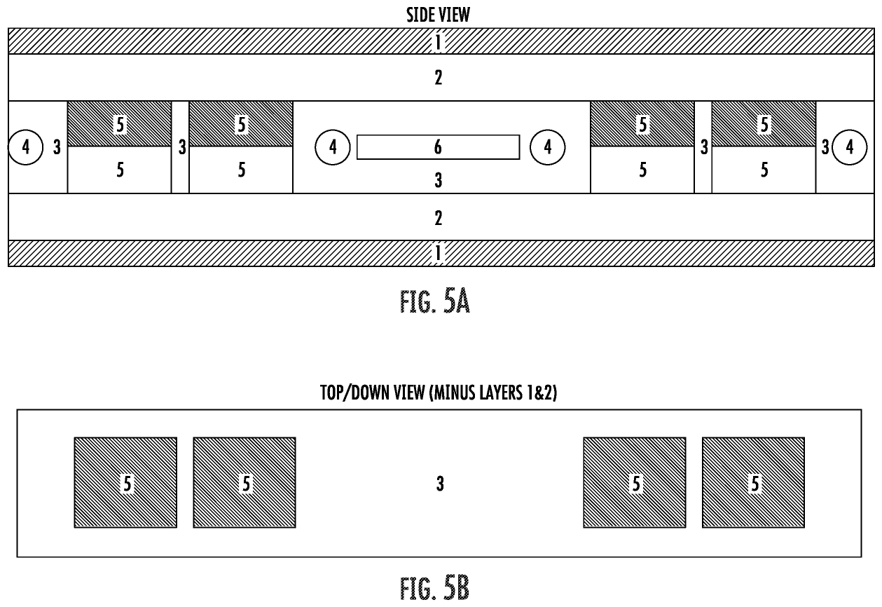

Referring now to FIGS. 5A and 5B, a cross section and top view, respectively, of a thermal regulating unit in accordance with some embodiments of the present inventive concept will be discussed. As illustrated in cross section of FIG. 5A, a layer of, for example, carbon fiber thermal conductive material 1 may be sealed to a waterproof thermal conductive layer 2. An insulating layer 3 may be provided between the waterproof thermal conductive layers 2. As illustrated in FIG. 5A, the insulating layer 3 includes a plurality of passive thermal sensors 4 and a plurality of pairs of coolers/heaters (Peltier coolers 5). As illustrated the coolers/heaters are positioned such that both a cool side and a hot side are accessible. As further illustrated, the insulating layer further includes a flexible circuit board. The circuit board 6 should have minimal impact to thermal conductivity. In some embodiments, the passive thermal sensors 4 and the power may be combined on a single circuit board. The entire thermal regulating unit is vacuumed sealed with, for example, a thermal conductive silicon.

FIG. 5B is a top down view of the insulating layer 3 having the Peltier coolers 5 positioned thereon. It will be understood that layers 1 and 2 of FIG. 5A have been removed in FIG. 5A. When all the air is removed from the between the layers, a thermally conductive adhesive must generally be used. However, electrically conductive adhesives should be avoided.

As discussed above, embodiments of the present inventive concept use thermally conductive material layers. This thermally conductive material may be any material capable of performing embodiments discussed herein. For example, one example material may be thermal grizzly Carbonaut (20 Watts). This material is thermally conductive but also electrically conductive. Thus, the circuits should be isolated from this layer. The material should be strong flexible and damage resistant. Another example material is Thermopad Thermal Grizzly Silicon (8 Watts). This material is weak, easily torn and easily molds/conforms to contours. The cooling/heating pad should be flexible enough to contour to the adjacent skin and provide as much skin contact as possible.

During an intense workout the human body can burn, for example, 800 to 1250 calories an hour. 2500 calories is equal to 120 Watts. The thermal layers in accordance with embodiments discussed herein need to transfer more than the body produces due to air gaps between skin and thermal pads. Generally, in some embodiments 1-2 Watts of power per minute is needed to maintain the temperature. Similarly, 4-8 Watts of power may be needed to cool or heat the user.

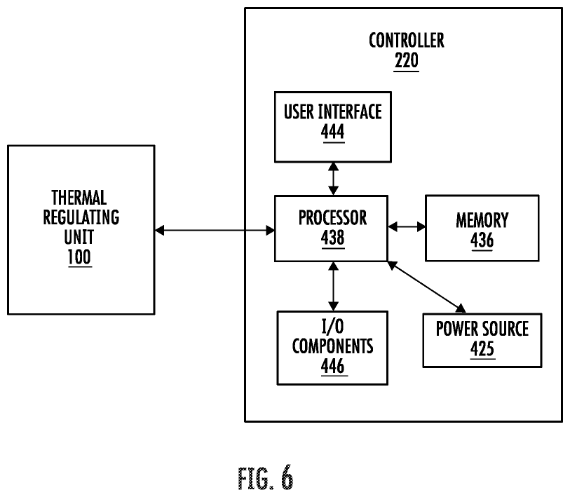

Referring now to FIG. 6, a block diagram illustrating a controller 220 in communication with a thermal regulating unit 100 in accordance with some embodiments of the present inventive concept will be discussed. As is clear from the embodiments discussed above, some aspects of the present inventive concept include the user of a controller 220. The controller 220 may be included at any module of the system without departing from the scope of the present inventive concept. However, as discussed above the controller 220 is discussed as a portable stand-alone module. Exemplary embodiments of a controller 220 configured in accordance with embodiments of the present inventive concept are discussed with respect to FIG. 6. The controller 220 may include a user interface 644, including, for example, input device(s) such as a keyboard or keypad, a display, a speaker and/or microphone, and a memory 636 that communicate with a processor 638. The controller 220 may further include I/O components 646 that also communicate with the processor 638. The I/O components 446 can be used to transfer information between the controller 220 and the thermal regulating units 100.

As briefly discussed above, some embodiments of the present inventive concept provide thermal regulating units that are positioned in garments to regulate temperature of the body. As discussed, thermal regulating units discussed herein are designed with several layers to accomplish longevity of the product, washability, rapid thermal dispersion, lower power consumption, and regulation of temperature across the major body heat sources. The design further provides a lightweight unit having reduced power consumption; high durability; and allows regulation of temperature up to +/−20 degrees of ambient temperature or more. The units may be removed from the garment such that the garment is machine washable.

The aforementioned flow logic and/or methods show the functionality and operation of various services and applications described herein. If embodied in software, each block may represent a module, segment, or portion of code that includes program instructions to implement the specified logical function(s). The program instructions may be embodied in the form of source code that includes human-readable statements written in a programming language or machine code that includes numerical instructions recognizable by a suitable execution system such as a processor in a computer system or other system. The machine code may be converted from the source code, etc. Other suitable types of code include compiled code, interpreted code, executable code, static code, dynamic code, object-oriented code, visual code, and the like. The examples are not limited in this context.

If embodied in hardware, each block may represent a circuit or a number of interconnected circuits to implement the specified logical function(s). A circuit can include any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Qualcomm® Snapdragon®; Intel® Celeron®, Core (2) Duo®, Core i3, Core i5, Core i7, Itanium®, Pentium®, Xeon®, Atom® and XScale® processors; and similar processors. Other types of multi-core processors and other multi-processor architectures may also be employed as part of the circuitry. According to some examples, circuitry may also include an application specific integrated circuit (ASIC) or a field programmable gate array (FPGA), and modules may be implemented as hardware elements of the ASIC or the FPGA. Furthermore, embodiments may be provided in the form of a chip, chipset or package.

Although the aforementioned flow logic and/or methods each show a specific order of execution, it is understood that the order of execution may differ from that which is depicted. Also, operations shown in succession in the flowcharts may be able to be executed concurrently or with partial concurrence. Furthermore, in some embodiments, one or more of the operations may be skipped or omitted. In addition, any number of counters, state variables, warning semaphores, or messages might be added to the logical flows or methods described herein, for purposes of enhanced utility, accounting, performance measurement, or providing troubleshooting aids, etc. It is understood that all such variations are within the scope of the present disclosure. Moreover, not all operations illustrated in a flow logic or method may be required for a novel implementation.

Where any operation or component discussed herein is implemented in the form of software, any one of a number of programming languages may be employed such as, for example, C, C++, C#, Objective C, Java, Javascript, Perl, PHP, Visual Basic, Python, Ruby, Delphi, Flash, or other programming languages. Software components are stored in a memory and are executable by a processor. In this respect, the term “executable” means a program file that is in a form that can ultimately be run by a processor. Examples of executable programs may be, for example, a compiled program that can be translated into machine code in a format that can be loaded into a random access portion of a memory and run by a processor, source code that may be expressed in proper format such as object code that is capable of being loaded into a random access portion of a memory and executed by a processor, or source code that may be interpreted by another executable program to generate instructions in a random access portion of a memory to be executed by a processor, etc. An executable program may be stored in any portion or component of a memory. In the context of the present disclosure, a “computer-readable medium” can be any medium (e.g., memory) that can contain, store, or maintain the logic or application described herein for use by or in connection with the instruction execution system.

A memory is defined herein as an article of manufacture and including volatile and/or non-volatile memory, removable and/or non-removable memory, erasable and/or non-erasable memory, writeable and/or re-writeable memory, and so forth. Volatile components are those that do not retain data values upon loss of power. Nonvolatile components are those that retain data upon a loss of power. Thus, a memory may include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), or other like memory device.

The devices described herein may include multiple processors and multiple memories that operate in parallel processing circuits, respectively. In such a case, a local interface, such as a communication bus, may facilitate communication between any two of the multiple processors, between any processor and any of the memories, or between any two of the memories, etc. A local interface may include additional systems designed to coordinate this communication, including, for example, performing load balancing. A processor may be of electrical or of some other available construction.

It should be emphasized that the above-described embodiments of the present disclosure are merely possible examples of implementations set forth for a clear understanding of the principles of the disclosure. It is, of course, not possible to describe every conceivable combination of components and/or methodologies, but one of ordinary skill in the art may recognize that many further combinations and permutations are possible. That is, many variations and modifications may be made to the above-described embodiment(s) without departing substantially from the spirit and principles of the disclosure. All such modifications and variations are intended to be included herein within the scope of this disclosure and protected by the following claims.

That which is claimed is:

1. A thermal regulating unit that regulates temperature of a body when positioned next to thermal points on the body, the thermal regulating unit comprising:
a first thermally conductive layer adjacent skin of the body and including embedded electrical wiring;
a first passive thermometer sensor integrated with the first thermally conductive layer;
a plurality of spaced-apart thermoelectric heaters and/or coolers on the first thermal conductivity layer;
a second thermally conductive layer opposite the first thermally conductive layer, the second thermally conductive layer being exposed to ambient air or fluid and including embedded electrical wiring;
a second passive thermometer sensor integrated with the second thermally conductive layer; and
a thermal insulating layer between the first and second thermally conductive layers to create a thermal barrier therebetween and adhere the first and second thermally conductive layers together,
wherein the thermal regulating unit operates responsive to a controller to regulate the temperature of the body.

2. The thermal regulating unit of claim 1, wherein the thermally conductive layer adjacent the skin of the body is in direct contact with the skin of the body.

3. The thermal regulating unit of claim 1, wherein the controller that regulates the temperature of the body is a separate, self-contained device that couples to the thermal regulating unit.

4. The thermal regulating unit of claim 3, further comprising a power source associated with the controller for supplying power to the thermal regulating unit,
wherein the controller and the power source are separate from the thermal regulating unit.

5. The thermal regulating unit of claim 1:
wherein the thermal regulating unit is positioned next to a thermal point of the body where veins are close to the skin;
wherein the first passive thermometer sensor and second passive thermometer sensor convert heat energy to electricity;
wherein the thermal regulating unit transmits a feedback signal to the controller; and
wherein the controller manages the power supplied to the thermal regulating unit based on the feedback signal.

6. The thermal regulating unit of claim 1:
wherein the thermal regulating unit is one of a plurality of thermal regulating units coupled to the controller and a power source associated with the controller; and
wherein the controller regulates the temperature of the body based on the feedback signals from the plurality of thermal regulating units.

7. The thermal regulating unit of claim 1, wherein the controller regulates the temperature of the body by increasing or decreasing the temperature in a range of 20 degrees Fahrenheit.

8. The thermal regulating unit of claim 1, wherein the thermal regulating unit is positioned in a close-fitting garment.

9. The thermal regulating unit of claim 8, wherein the thermal regulating unit is positioned in the close-fitting garment using integrated pockets in the close-fitting garment that allow the thermal regulating unit to be inserted therein and removed therefrom.

10. The thermal regulating unit of claim 8, wherein thermal regulating units is affixed to the garment using a fastener.

11. The thermal regulating unit of claim 8, wherein the close-fitting garment is related to military, sports, and/or daily wear activities.

12. The thermal regulating unit of claim 1, wherein the thermal regulating unit is waterproof and washable.

13. The thermal regulating unit of claim 1, wherein the body is a body of a living mammal.

14. The thermal regulating unit of claim 13, wherein the thermal regulating unit is positioned next to one or more thermal points on the body of the living mammal.

15. A system for actively regulating temperature of a body, the system comprising:
a garment;
one or more thermal regulating units positioned on the garment, each of the thermal regulating units comprising:
a first thermally conductive layer adjacent skin of the body and including embedded electrical wiring;
a first passive thermometer sensor integrated with the first thermally conductive layer;
a plurality of spaced-apart thermoelectric coolers on the first thermal conductivity layer;
a second thermally conductive layer opposite the first thermally conductive layer, the second thermally conductive layer being exposed to ambient air or fluid and including embedded electrical wiring;
a second passive thermometer sensor integrated with the second thermally conductive layer; and
a thermal insulating layer between the first and second thermally conductive layers to create a thermal barrier therebetween and adhere the first and second thermally conductive layers together;
a controller that regulates the temperature of the body based on feedback signals from the one or more thermal regulating units; and
a power source coupled to the controller to provide power to the system.

16. The system of claim 15, wherein the first thermally conductive layer adjacent the skin of the body is in direct contact with the skin of the body.

17. The system of claim 15, wherein the controller to regulate the temperature of the body is a separate, self-contained device that communicates with one or more of the thermal regulating units.

18. The system of claim 17, wherein the controller and the power source are an integrated unit separate from the or more thermal regulating units.

19. The system of claim 15:
wherein the one or more thermal regulating units are positioned next to a thermal point of the body where veins are close to the skin;
wherein the first passive thermometer sensor and second passive thermometer sensor convert heat energy to electricity;
wherein one or more the thermal regulating units transmit a feedback signal to the controller; and
wherein the controller manages the power supplied to the one or more thermal regulating units based on the feedback signal.

20. The system of claim 15:
wherein the one or more thermal regulating units are a plurality of thermal regulating units; and
wherein the plurality of thermal regulating units are coupled to corresponding ones of a plurality of controllers that regulate the temperature of the body based on the feedback signals from the corresponding ones of the plurality of thermal regulating units.

21. The system of claim 15, wherein the one or more thermal regulating units are positioned in a close-fitting garment.

22. The system of claim 21, wherein the one or more thermal regulating units are positioned in the close-fitting garment using integrated pockets in the close-fitting garment that allow the one or more thermal regulating units to be removed therefrom and inserted therein.

23. The system of claim 21, wherein the one or more thermal regulating units are affixed to the garment using a fastener.

24. The system of claim 21, wherein the close-fitting garment is worn during military, sports, and/or daily wear activities.

25. The system of claim 15, wherein the one or more thermal regulating units are waterproof and washable.

26. The system of claim 15, wherein the body is a body of a living mammal.

27. The system of claim 26, wherein the one or more thermal regulating units are positioned next to thermal points on the body of the living mammal.

28. A garment having one more thermal regulating units positioned therein, the garment comprising:
a fastening means;
one or more thermal regulating units affixed to the garment using the fastening means, the one or more thermal regulating units each comprising:
a first thermally conductive layer adjacent skin of a body and including embedded electrical wiring;
a first passive thermometer sensor integrated with the first thermally conductive layer;
a plurality of spaced-apart thermoelectric coolers on the first thermal conductivity layer;
a second thermally conductive layer opposite the first thermally conductive layer, the second thermally conductive layer being exposed to ambient air or fluid and including embedded electrical wiring;
a second passive thermometer sensor integrated with the second thermally conductive layer; and
a thermal insulating layer between the first and second thermally conductive layers to create a thermal barrier therebetween and adhere the first and second thermally conductive layers together,
wherein the thermal regulating unit operates responsive to a controller to regulate a temperature of the body.
